(19) United States
(12) Patent Application Publication
(10) Pub. No.: US 2018/0043043 A1
(43) Pub. Date: Feb. 15, 2018

(54) BANDAGE HAVING A COMPACT LED INSERT

(71) Applicant: Donald SPECTOR, New York, NY (US)

(72) Inventor: Donald SPECTOR, New York, NY (US)

(21) Appl. No.: 15/284,776

(22) Filed: Oct. 4, 2016

Related U.S. Application Data
(60) Provisional application No. 62/374,027, filed on Aug. 12, 2016.

(51) Int. Cl. A61L 2/10; A61F 13/02; A61N 5/06; A61F 13/00

(52) U.S. Cl. CPC A61L 2/10; A61F 13/00063; A61F 13/0206; A61N 5/0624; A61N 5/0625; A61N 2005/0645; A61N 2005/0651; A61N 2005/0659; A61N 2005/0661; A61L 2202/11

(57) ABSTRACT

A portable capsule for insertion into a bandage consists of an light emitting diode (LED) connected to a power source encased in a housing. The housing has a clear window that allows the UV light from the LED to shine through and sterilize the surrounding bandage area. The capsule is inserted into the bandage material and held within the bandage to kill any bacteria that enters the bandage. The capsule is kept away from any direct skin contact, so there is no risk of burns or skin damage from the UV light. When the bandage is removed from the patient, the capsule can be removed from the bandage, cleaned, and re-used in the next bandage.

### CROSS REFERENCE TO RELATED APPLICATIONS

Applicant claims priority under 35 U.S.C. §119(e) of U.S. Provisional Patent Application Ser. No. 62/374,027 filed Aug. 12, 2016, the disclosure of which is incorporated by reference.

### BACKGROUND OF THE INVENTION

### 1. Field of the Invention

This invention relates to a compact insert that can be placed inside a bandage for disinfecting the bandage while in use.

### 2. The Prior Art

Disposable bandages often can harbor bacteria while being worn, thus increasing the risk of infection. Often, the use of antibiotic ointments is not sufficient to kill the bacteria that can cause the infection. U.S. Pat. No. 8,372,128 has attempted to solve this problem by incorporating a UV light emitter into the bandage. However, this solution is expensive, as the entire bandage with the UV light source is discarded after use.

### SUMMARY OF THE INVENTION

It is therefore an object of the invention to provide a way to incorporate a UV light emitter into a bandage in a simple and economical manner.

This and other objects are accomplished by a portable capsule containing a battery and a UV light source, all contained in a small, flat housing. The capsule is then inserted into the bandage material and held within the bandage to kill any bacteria that enters the bandage. The capsule is kept away from any direct skin contact, so there is no risk of burns or skin damage from the UV light. The invention also consists of a bandage that is configured to receive the capsule via an opening in the side of the bandage. The capsule is then slid in between the layers of gauze or on tope of the gauze layer and can be held in place by a closure element, which can be separate or part of the bandage. When the bandage is removed from the patient, the capsule can be removed from the bandage, cleaned, and re-used in the next bandage.

The UV capsule consists of one or more light emitting diodes (LEDs) that emit light in the UV spectrum, connected to a power source in the form of a disposable or reusable battery, all encased in a housing. The housing can be made of plastic or other suitable material that can be easily cleaned and/or sterilized between uses. The housing has a clear window that allows the UV light from the LEDs to shine through and sterilize the surrounding bandage area.

In another embodiment, the LED delivers infrared light, and the capsule is placed so that the light directly contacts the skin when the bandage is placed. This has the effect of promoting muscle healing.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a front view of the capsule according to the invention;

FIG. 2 shows a side view of the capsule;

FIG. 3 shows a side view of the capsule inserted into a bandage;

FIG. 4 shows a top view of the bandage; and

FIG. 5 shows a side view of an alternative embodiment of the capsule and bandage.

### DETAILED DESCRIPTION OF THE PREFERRED EMBODIMENT

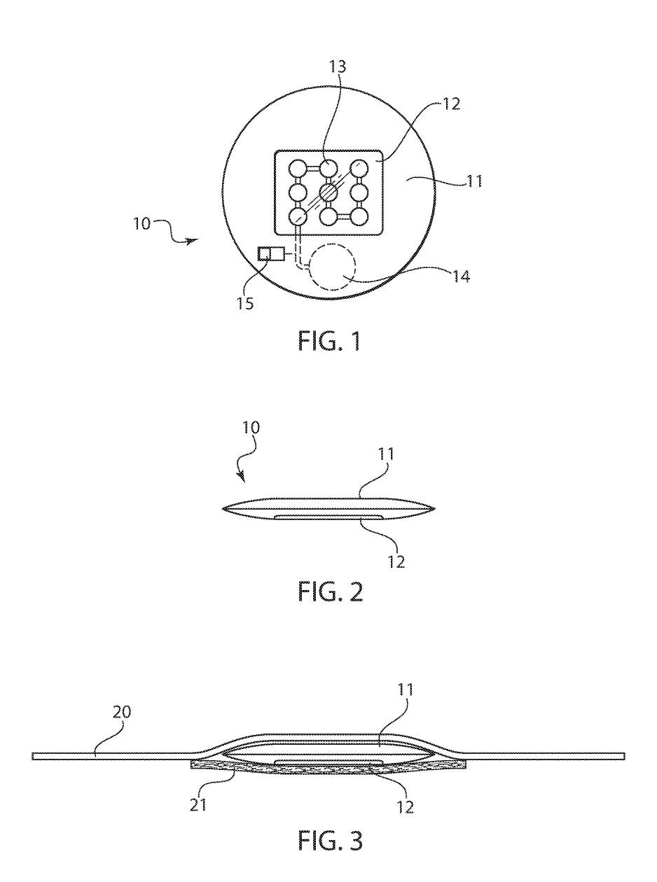

Referring now in detail to the drawings, FIGS. 1 and 2 show a capsule 10 according to the invention, which comprises a plastic housing 11 with a translucent window 12. Housing 11 could also be made entirely translucent to eliminate the need to assemble a window. Inside housing 11 are a plurality of small LED bulbs 13 connected to a power source in the form of a battery 14. LED bulbs 13 emit UV light. Housing 11 can be configured to be openable so that battery 14 can be replaced when it dies, or can be configured permanently sealed so that capsule 10 is discarded when the battery dies. Housing 11 can be made of rigid or flexible material and is configured to be watertight so that moisture does not reach the LEDs 13 or battery 14.

Capsule 10 can be round, square or any desired shape. It is sized so that it can fit into a bandage 20, as shown in FIG. 3. Window 12 is arranged facing downward onto gauze 21, so that the UV light from bulbs 13 passes through window 12 and can disinfect gauze 13 during use. Switch 15 can be used to disconnect battery 14 from LEDs 13 in order to turn the capsule on and off. The capsule 10 is generally turned on prior to insertion into bandage 20 and then is turned off after the capsule 10 is removed and bandage 20 is discarded.

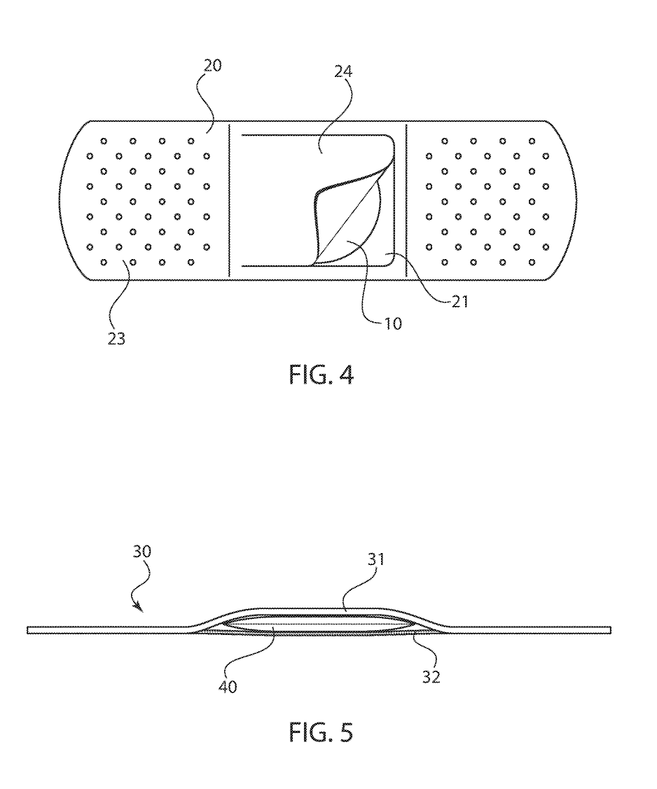

FIG. 4 shows a top view of bandage 20, which consists of a center portion containing gauze 21, and two adhesive sections 23. A flap 24 is located in the center portion and creates an opening to allow for insertion of capsule 10. Flap 24 can have adhesive around its edges so as to seal capsule 10 in bandage 20 during use. After use, capsule 10 is removed from bandage 20, bandage 20 is discarded, and capsule 10 is then cleaned and/or sterilized before being used again.

During use, UV light from LEDs 13 acts on gauze 21 and kills any bacteria that enter gauze 21, thus keeping bandage 20 sterile during use and preventing wound infection.

FIG. 5 shows an alternative embodiment of the invention. Here, capsule 40 has LEDs that emit IR light (not shown) rather than UV light. In this embodiment, capsule 40 is placed in between a bottom translucent layer 32 and a top layer 31 of the bandage so that light from the IR source can travel through translucent layer 32 and into the skin and muscles below, in order to heal injured muscles. As with the embodiment of FIGS. 1-4, capsule 40 can be removed from bandage 30 after use, cleaned and then used again.

Capsule 40 can be used with disposable bandages, or can be affixed to the body using re-usable cloth bandages as well.

Accordingly, while only a few embodiments of the present invention have been shown and described, it is obvious that many changes and modifications may be made thereunto without departing from the spirit and scope of the invention.

What is claimed is:

1. A bandage system comprising:
a disinfecting capsule comprising:
a housing;
a power source disposed in the housing; and
a light source connected to the power source and disposed in the housing; and
a bandage having an opening and a compartment for receiving the disinfecting capsule.

2. The bandage system according to claim 1, wherein the light source transmits UV light and wherein the capsule is configured so that the UV light is transmitted through the capsule.

3. The bandage system according to claim 1, further comprising a switch to turn the light source on and off.

4. The bandage system according to claim 1, wherein the housing is waterproof.

5. The bandage system according to claim 1, wherein the power source is a battery.

6. The bandage system according to claim 5, wherein the capsule is configured so that the battery is replaceable.

7. The bandage system according to claim 1, wherein the compartment is configured so that the capsule when inserted in the bandage is disposed adjacent absorbent material in the bandage.

8. The bandage system according to claim 1, wherein the light source comprises at least one light emitting diode (LED).

8. The bandage system according to claim 1, wherein the light source emits light in the infrared range.

9. The bandage system according to claim 1, wherein the compartment is configured so that the capsule when inserted allows the light from the light source to be transmitted through the bandage.

10. The bandage system according to claim 1, wherein the capsule has a window formed of transparent material adjacent the light source to allow light to be transmitted through the capsule.
